(19) United States
(12) Patent Application Publication
(10) Pub. No.: US 2025/0169684 A1
(43) Pub. Date: May 29, 2025

(54) Distributed Bending and Mode Control for Bendable Medical Devices

(71) Applicants: CANON U.S.A., INC., MELVILLE, NY (US); THE GENERAL HOSPITAL CORPORATION, BOSTON, MA (US)

(72) Inventors: Charles George Hwang, Wellesley, MA (US); Brian Ninni, Woburn, MA (US); Takahisa Kato, Brookline, MA (US); Yolonda Lorig Colson, Dover, MA (US); Jorind Beqari, Boston, MA (US)

(21) Appl. No.: 18/958,472

(22) Filed: Nov. 25, 2024

Related U.S. Application Data
(60) Provisional application No. 63/603,384, filed on Nov. 28, 2023.

(51) Int. Cl. A61B 1/005; A61B 1/00; A61B 1/018; A61B 10/04

(52) U.S. Cl. CPC A61B 1/0055; A61B 1/00045; A61B 1/018; A61B 10/04

(57) ABSTRACT

Provided herein is a robot apparatus, system and method of use. The robot system may comprise a bendable body having a plurality of steerable segments, a display, and a controller, where the controller controls actuation of the steerable segments based on an input data. The controller may provide a display that provides information to the user about the various bending modes, and the controller may change the bending mode based on information from the bendable body.

### CROSS-REFERENCE TO RELATED APPLICATIONS

This application claims priority to provisional application Ser. No. 63/603,384 filed 28 Nov. 2023, herein incorporated by reference in its entirety.

### BACKGROUND

### Field of the Disclosure

The present disclosure relates to medical devices. More particularly, the disclosure exemplifies embodiments of a steerable medical device, such as endoscopes or catheters that has multiple bendable sections. The steerable medical device has different modes of operation and the switching between these modes can be controlled automatically or by the user.

### Description of the Related Art

Endoscopy, bronchoscopy, catheterization, and other medical procedures facilitate the ability to look inside a body. During such a procedure, a flexible medical device may be inserted into a patient's body, and an instrument may be passed through the device to examine or treat an area inside the body. For viewing and/or treating the airway of a patient, a bronchoscope may be used. Medical tools such as cameras and biopsy needles may be inserted through a tool channel in the bronchoscope to target area in the patient for diagnosis, planning, medical procedure(s), treatment, etc.

Some of these medical devices are guided through a disposable or limited-use flexible tubular body often referred to as a sleeve or sheath or introducer sheath. Some of these introducer sheaths or sleeves are robotically controlled. A robotically controlled catheter or endoscope has a catheter sheath with a steerable distal section and a non-steerable proximal section. The proximal section connects to an actuator unit via an electromechanical connector, and the distal section is sized to be introduced into a patient's anatomy through natural orifices or small surgical incisions. Similarly, catheters and endoscopes that are insertable into a patient can be operated manually by a user without automated or robotic control. In either case, one or more channels extend along a central lumen of the sheath to allow access for imaging devices (miniature cameras or optical fiber probes) and/or end effectors (biopsy tools or therapy probes), and/or for passing fluids (contrast agents, gas, or flushing solutions).

Most catheters on the market today have a single steerable distal section, where the user has the ability to direct the bending motion of this steerable distal section. Various modes of operation of these catheters are described in, for example, U.S. Pat. No. 10,765,303 which provides a mode that changes the bend of a curve when a force threshold is met.

A new catheter with three steerable sections at the distal end of the catheter is in development. These steerable distal sections can be bent independently, and different operation modes are available. For example, the distal-most bending segment may be bent while the other sections are left unbend in the Tip Bending mode of operation that is particularly useful for navigation of the steerable device in a lumen. See, for example, U.S. Pat. No. 11,278,366. In another mode, only the middle segment is bent (called Targeting mode, or Chickenhead mode since this movement looks like a chicken hunting for seeds on the ground - - - or more relevantly, a biopsy tool hunting for various biopsy locations. These modes are described in, for example, U.S. Pat. No. 11,096,552, herein incorporated by reference in its entirety.

However, additional modes optimizing the scope of movement allowed by a catheter having multiple distal bending sections (either the three exemplified in the above references or two, four, five, or more sections) have not been fully explored. Additionally, the transitions between the varying control modes have not been sufficiently addressed. Additionally, the ability to change modes upon need-such as when a tool needs to be passed through a tightly curved catheter, has not been fully explored.

Thus, there is needed a flexible medical device that overcomes the deficiencies discussed above.

### SUMMARY

Accordingly, it is a broad object of the invention to provide a steerable medical device and a method of viewing and switching between the various modes of the steerable device.

According to some embodiments, there is provided a medical system. This medical system has a bendable body such as an endoscope or catheter having a steerable distal section comprising a plurality of steerable segments and a tool channel configured to hold a medical tool or a camera that extends through the steerable distal section; a display device; and a controller. The controller is configured to: control the steerable distal section in a first bending mode such as Tip Bending mode or Targeting mode and in a distributed bending mode and switch between the first bending mode and the distributed bending mode.

The switching function is configured to perform at least one of:
(a) be activated by a user, and wherein the controller is further configured to display on the display device an indicator of a current mode and an indicator that the mode should be changed into or out of the distributed bending mode,
(b) be activated by a user, and wherein the controller is further configured to display a notification on the display device that the steerable distal section has reached a location within a distance D of a region of interest, or when a bend status is above a bend threshold T,
(c) be activated when the controller detects a camera or a biopsy tool being removed from the bendable body, and/or
(d) be activated when controller receives information that the steerable distal section has reached a location within a distance D of a region of interest, or when the bend status is above a bend threshold T.

In some embodiments of the invention, there is provide a method of performing a procedure using a bendable body comprising: navigating towards a target in a first bending mode with a camera in the tool channel; changing to distributed bending mode; removing the camera; inserting a tool; changing to the first bending mode; and performing a procedure.

Further features of the present disclosure will become apparent from the following description of exemplary embodiments with reference to the attached drawings, where like structure is indicated with like reference numerals.

### BRIEF DESCRIPTION OF THE DRAWINGS

For the purposes of illustrating various aspects of the disclosure, wherein like numerals indicate like elements, there are shown in the drawings simplified forms that may be employed, it being understood, however, that the disclosure is not limited by or to the precise arrangements and instrumentalities shown. To assist those of ordinary skill in the relevant art in making and using the subject matter hereof, reference is made to the appended drawings and figures.

FIG. 1 illustrates at least one embodiment of an imaging, continuum robot, or endoscopic apparatus or system in accordance with one or more aspects of the present disclosure.

FIG. 2 is a schematic diagram showing at least one embodiment of an imaging, bendable body, or continuum robot apparatus or system in accordance with one or more aspects of the present disclosure;

FIG. 3 illustrate at least one embodiment example of a continuum robot and/or medical device that may be used with one or more technique(s), including autonomous navigation technique(s), in accordance with one or more aspects of the present disclosure. Detail A illustrates one guide ring of the bendable body.

FIG. 4 is a schematic diagram showing at least one embodiment of an imaging, continuum robot, bendable body, or endoscopic apparatus or system in accordance with one or more aspects of the present disclosure.

FIG. 5 is a schematic diagram showing at least one embodiment of a console or computer that may be used with one or more autonomous navigation technique(s) in accordance with one or more aspects of the present disclosure.

FIGS. 6(A)-6(C) illustrate one or more principles of catheter or continuum robot tip manipulation by actuating one or more bending segments of a continuum robot or bendable body 104 of FIGS. 6(A)-6(C) in accordance with one or more aspects of the present disclosure.

FIG. 7 illustrates Uniform Curvature based on Section Lengths.

FIG. 8 illustrates a pose having a uniform curvature distribution at 90 degrees that is moved by a uniform curvature distribution at 30 degrees.

FIG. 9 illustrates the transitioning into a uniform curvature distribution.

FIGS. 10(A) and 10(B) illustrates maintaining local angle to while still changing orientation.

FIG. 11 illustrates counteractions that can be taken in a pose having a bendable middle segment to remove effects of crosstalk to maintain output orientation.

FIGS. 12(A) and 12(B) illustrate a movement mode that allows for smaller movements of the middle and proximal segments, relative to tip movement.

FIGS. 13(A)-13(D) illustrates an exemplary display with a three-section bendable body in tip bending mode (FIG. 13(A) and FIG. 13(B)) or in distributed bending mode (FIG. 13(C) and FIG. 13(D).

FIG. 14 provides an exemplary workflow for camera in mode change sequence.

FIG. 15 provides an exemplary workflow for a change sequence for a simple tool pass.

FIG. 16(A)-16(E) illustrate visualizations of a three-section robot in different bending modes.

FIG. 17(A) and FIG. 17(B) illustrate the potential ability and problem for passing a tool through the robot.

FIG. 18 illustrates an exemplary interface where passable tools are listed.

Throughout the figures, the same reference numerals and characters, unless otherwise stated, are used to denote like features, elements, components or portions of the illustrated embodiments. Moreover, while the subject disclosure will now be described in detail with reference to the figures, it is done so in connection with the illustrative exemplary embodiments. It is intended that changes and modifications can be made to the described exemplary embodiments without departing from the true scope and spirit of the subject disclosure as defined by the appended claims.

### DESCRIPTION OF THE EMBODIMENTS

The present disclosure has several embodiments and relies on patents, patent applications and other references for details known to those of the art. Therefore, when a patent, patent application, or other reference is cited or repeated herein, it should be understood that it is incorporated by reference in its entirety for all purposes as well as for the proposition that is recited.

### <Robotic Catheter System>

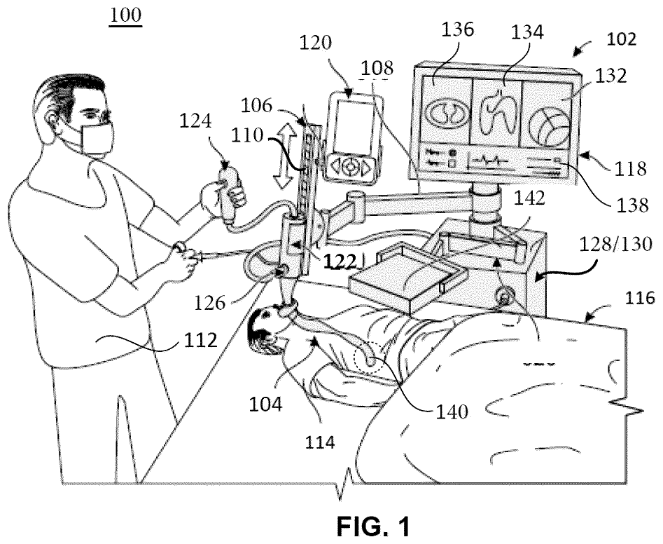

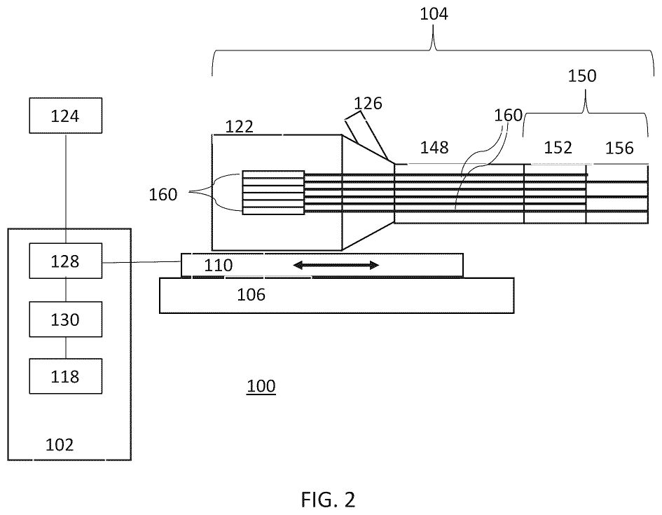

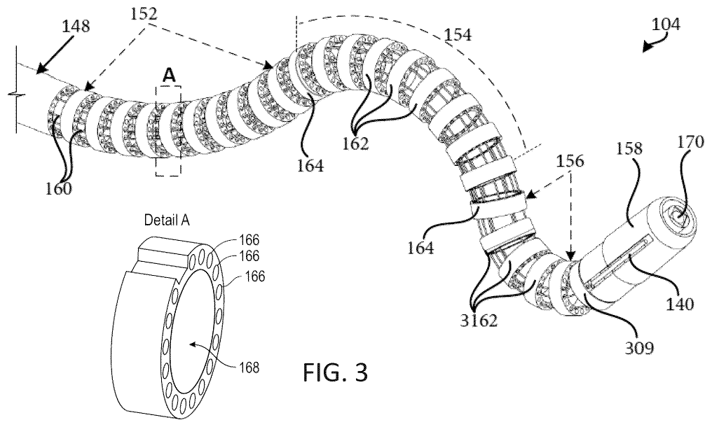

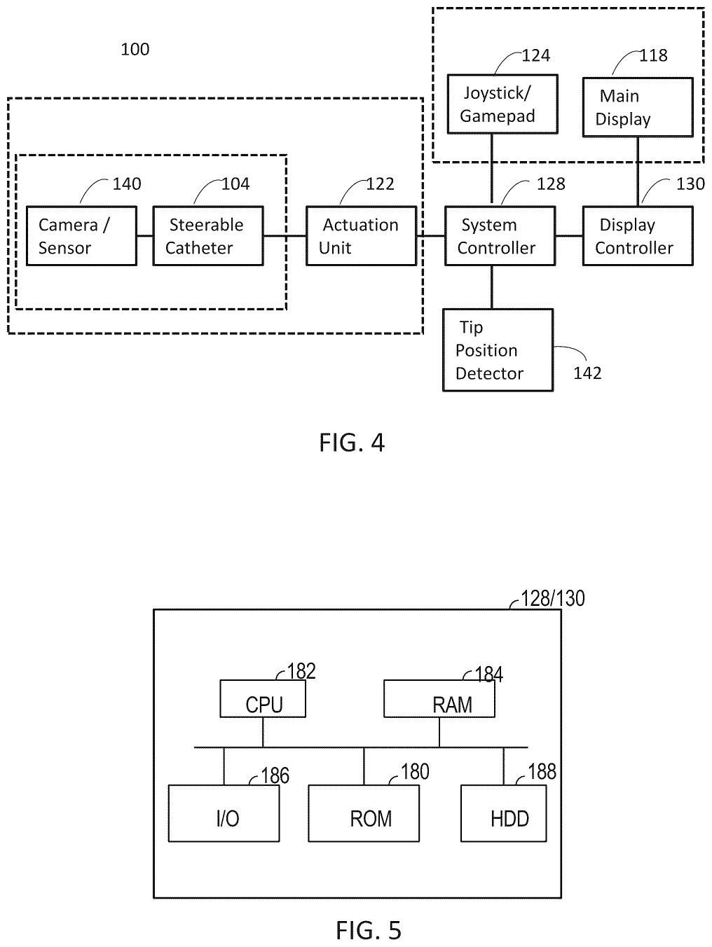

An embodiment of a robotic catheter system 100 is described in reference to FIG. 1 through FIG. 4. FIG. 1 illustrates a simplified representation of a medical environment, such as an operating room, where a robotic catheter system 100 can be used. FIG. 2 illustrates a functional block diagram of the robotic catheter system 100. FIGS. 3A-C represents the catheter and bending. FIGS. 4-5 illustrates a logical block diagram of the robotic catheter system 100. In this example, the system 100 includes a system console 102 (computer cart) operatively connected to a bendable body 104 via a robotic platform 106. The robotic platform 106 includes one or more than one robotic arm 108 and a linear translation stage 110.

In FIG. 1, A user 112 (e.g., a physician) controls the robotic catheter system 100 via a user interface unit (operation unit) to perform an intraluminal procedure on a patient 114 positioned on an operating table 116. The user interface may include at least one of a main display 118 (a first user interface unit), a secondary display 120 (a second user interface unit), and a handheld controller 124 (a third user interface unit). The main display 118 may include, for example, a large display screen attached to the system console 102 or mounted on a wall of the operating room and may be, for example, designed as part of the robotic catheter system 100 or be part of the operating room equipment. Optionally, there is a secondary display 120 that is a compact (portable) display device configured to be removably attached to the robotic platform 106. Examples of the secondary display 120 include a portable tablet computer or a mobile communication device (a cellphone).

The bendable body 104 may be steerable and is actuated via an actuator unit 122. The actuator unit 103 is removably attached to the linear translation stage 110 of the robotic platform 106. The handheld controller 124 may include a gamepad-like controller with a joystick having shift levers and/or push buttons. It may be a one-handed controller or a two-handed controller. In one embodiment, the actuator unit 122 is enclosed in a housing having a shape of a catheter handle. One or more access ports 126 are provided in or around the catheter handle. The access port 126 is used for inserting and/or withdrawing end effector tools and/or fluids when performing an interventional procedure of the patient 114.

In some embodiments, the handheld controller 124 and/or an integrated controller and/or the GUI have inputs to modify the mode or settings. For example, a change between “Tip Bending” and “Proximal Bending” can be toggled with the push of a button (or similar digital/on-off method). In one scenario, the mode will cycle between the two (or more) modes each time the ‘button’ is pressed & released. If there are more than two modes, a method to reverse the direction of the cycling can also be included.

In another scenario, the mode can be toggled based on the state of a digital/on-off input. This input can be something that maintains its state after being changed (like a switch or checkbox) or something that will revert back to the initial state once it is release (i.e. a push button). For example, the mode can be in “Tip Bending” when this state is OFF, and it will switch to “Proximal Bending” when the state is ON. If there are more than two modes, a ‘multi-state’ input could be implemented, or the mode can be determined based on the combined states of multiple inputs.

The amount of bending applied to each section when in distributed mode can also be adjusted with physical or virtual inputs. In one scenario, the ability to include a section in the distribution can be toggled on/off using manner described in the above paragraph.

In another scenario, the amount of distribution applied to each section can be adjusted. This can be provided with a range input element such as a slider or knob (real or virtual). For example, there can a virtual slider or number box which represents the percentage of the total distribution that is applied to the section. Alternatively, the value of this input can represent the reduction of distribution. For example, a value representing 25% will in turn result in distributing 75% of the input bending amount to the corresponding section. Similarly, these values can be adjusted using physical inputs. These inputs can be analog where the value within its range corresponds to the percentage (or reduction) of distribution of the corresponding section. This input can maintain its state when changed (i.e. a dial) or it can return to its starting state when released (i.e. a trigger). The range input element that is a physical device can also ‘adjust’ the distribution ratio, rather than directly represent it. For example, a press of a button or a push of an analog stick can increase or decrease the distribution value for the corresponding section. The rate at which it changes can be either fixed, modifiable, or be related to the value of the input (if it is analog).

If there are multiple sections, each section can have its own dedicated input. Alternatively, one input can correspond to multiple sections. In this scenario, they could both be affected in the same manner (i.e., both reduced or increased) or inversely, where one is increased and the other is decreased (or a combination of each if more than 2). This input can be a direct mapping, or an increment/decrement. If the input is an increment/decrement, the rate of increase/decrease could be the same for each or could be different for each. The adjustment of the rate of each can be modified in similar manners as described in earlier paragraphs.

The system console 102 includes a system controller 128, a display controller 130, and the main display 118. The main display 118 may include a conventional display device such as a liquid crystal display (LCD), an OLED display, a QLED display or the like. The main display 118 provides a graphic interface unit (GUI) configured to display one or more views. These views include one or more of a live view image 132, an intraoperative image 134, and a preoperative image 136, and other procedural information 138. Other views that may be displayed include a model view, a navigational information view, and/or a composite view. The live image view 132 may be an image from a camera or sensor 140 at the tip of the catheter. This view may also include, for example, information about the perception and navigation of the catheter 104. The preoperative image 136 may include pre-acquired 3D or 2D medical images of the patient acquired by conventional imaging modalities such as computer tomography (CT), magnetic resonance imaging (MRI), or ultrasound imaging. The intraoperative image 134 may include images used for image guided procedure such images may be acquired by fluoroscopy or CT imaging modalities. Intraoperative image 134 may be augmented, combined, or correlated with information obtained from a sensor, camera image, or catheter data.

In the various embodiments the camera or sensor 140 is a camera that is located at the distal end of the catheter. The camera 140 may be attached to the distal end of the catheter or it may be removably place using the tool channel. When a camera is used without an additional sensor, the tip position detector 142 is not needed.

In some embodiments, the camera or sensor is a catheter tip tracking sensor, the sensor may be located at the distal end of the catheter. The catheter tip tracking sensor 140 may be, for example, an electromagnetic (EM) sensor. If an EM sensor is used, a catheter tip position detector 142 is included in the robotic catheter system 100; this catheter tip position detector would include an EM field generator operatively connected to the system controller 128. Suitable electromagnetic sensors for use with a bendable body are well-known and described, for example, in U.S. Pat. No. 6,201,387 and international publication WO2020194212A1.

In various embodiments, a catheter tip tracking sensor 140 is not used. In these embodiments, the catheter tip position detector 142 is not needed.

Similar to FIG. 1, the diagram of FIG. 2 illustrates the robotic catheter system 100 includes the system controller 128 operatively connected to the display controller 130, which is connected to the display unit 118, and to the handheld control 124. The system controller 128 is also connected to the actuator unit 122 via the robotic platform 106, which includes the linear translation stage 110. The actuator unit 122 includes a plurality of motors 144 that control the plurality of drive wires 160. These drive wires travel through the bendable body 104. One or more access ports 126 may be located on the catheter. The catheter includes a proximal section 148 located between the actuator and the proximal bending section 152 where they actuate the proximal bending section. Three of the six drive wires 160 continue through the distal bending section 156 where they actuate this section and allow for a range of movement. This figure is shown with two bendable sections (152 and 156). Other embodiments as described herein can have three bendable sections (see FIG. 3). In some embodiments, a single bending section may be provided, or alternatively, four or more bendable sections may be present in the catheter.

FIG. 3 shows an exemplary embodiment of a bendable body 104. The bendable body 104 includes a non-steerable proximal section 148, a steerable distal section 150, and a catheter tip 158. The proximal section 148 and distal bendable section 150 (including separate sections 152, 154 and 156) are joined to each other by a plurality of drive wires 160 arranged along the wall of the catheter. The proximal section 148 is configured with thru-holes or grooves or conduits to pass drive wires 160 from the distal section 150 to the actuator unit 122. The distal section 150 is comprised of a plurality of bending segments including a distal segment 156, a middle segment 154, and a proximal segment 152. Each bending segment is bent by actuation of at least some of the plurality of drive wires 160 (driving members). The posture of the catheter may be supported by non-illustrated supporting wires (support members) also arranged along the wall of the catheter (see U.S. Pat. Pub. US2021/0308423). The proximal ends of drive wires 160 are connected to individual actuators or motors 144 of the actuator unit 122, while the distal ends of the drive wires 160 are selectively anchored to anchor members in the different bending segments of the distal bendable section 150.

Each bending segment is formed by a plurality of ring-shaped components (rings) with thru-holes, grooves, or conduits along the wall of the rings. The ring-shaped components are defined as wire-guiding members 162 or anchor members 164 depending on their function within the catheter. Anchor members 164 are ring-shaped components onto which the distal end of one or more drive wires 160 are attached. Wire-guiding members 162 are ring-shaped components through which some drive wires 160 slide through (without being attached thereto).

Detail “A” in FIG. 3 illustrates an exemplary embodiment of a ring-shaped component (a wire-guiding member 162 or an anchor member 164). Each ring-shaped component includes a central opening which forms the tool channel 168, and plural conduits 166 (grooves, sub-channels, or thru-holes) arranged lengthwise equidistant from the central opening along the annular wall of each ring-shaped component. Inside the ring-shaped component, an inner cover such as is described in U.S. Pat. Pub US2021/0369085 and US2022/0126060, may be included to provide a smooth inner channel and provide protection. The non-steerable proximal section 148 is a flexible tubular shaft and can be made of extruded polymer material. The tubular shaft of the proximal section 148 also has a central opening or tool channel 168 and plural conduits 166 along the wall of the shaft surrounding the tool channel 168. An outer sheath may cover the tubular shaft and the steerable section 150. In this manner, at least one tool channel 168 formed inside the bendable body 104 provides passage for an imaging device and/or end effector tools from the insertion port 126 to the distal end of the bendable body 104.

The actuator unit 122 includes one or more servo motors or piezoelectric actuators. The actuator unit 122 bends one or more of the bending segments of the catheter by applying a pushing and/or pulling force to the drive wires 160. As shown in FIG. 3A, each of the three bendable segments of the bendable body 104 has a plurality of drive wires 160. If each bendable segment is actuated by three drive wires 160, the bendable body 104 has nine driving wires arranged along the wall of the catheter. Each bendable segment of the catheter is bent by the actuator unit 122 by pushing or pulling at least one of these nine drive wires 160. Force is applied to each individual drive wire in order to manipulate/steer the catheter to a desired pose. Thus, any segment may be moved by moving at least one of the three drive wires anchored at that segment, and moving at least one drive wire of a segment means that one or more of the drive wires is moved. This can comprise pulling (or pushing) only one of the drive wires, or pulling one while simultaneously pushing on the other(s), etc.

The actuator unit 122 assembled with bendable body 104 is mounted on the linear translation stage 110. Linear translation stage 110 includes a slider and a linear motor. In other words, the linear translation stage 110 is motorized, and can be controlled by the system controller 128 to insert and remove the bendable body 104 to/from the patient's bodily lumen.

An imaging device 170 that can be inserted through the tool channel 168 includes an endoscope camera (videoscope) along with illumination optics (e.g., optical fibers or LEDs). The illumination optics provides light to irradiate the lumen and/or a lesion target which is a region of interest within the patient. End effector tools refer endoscopic surgical tools including clamps, graspers, scissors, staplers, ablation or biopsy needles, and other similar tools, which serve to manipulate body parts (organs or tumorous tissue) during examination or surgery. The imaging device 170 may be what is commonly known as a chip-on-tip camera and may be color or black-and-white.

In some embodiments, a tracking sensor 140 (e.g., an EM tracking sensor) is attached to the catheter tip 158. In this embodiment, bendable body 104 and the tracking sensor 140 can be tracked by the tip position detector 142. Specifically, the tip position detector 142 detects a position of the tracking sensor 140, and outputs the detected positional information to the system controller 100. The system controller 128, receives the positional information from the tip position detector 142, and continuously records and displays the position of the bendable body 104 with respect to the patient's coordinate system. The system controller 128 controls the actuator unit 122 and the linear translation stage 110 in accordance with the manipulation commands input by the user 112 via one or more of the user interface units (the handheld controller 124, a GUI at the main display 118 or touchscreen buttons at the secondary display 120).

The robotic medical system as described herein can include a continuum or multi-segment robot configured to form a curved geometry in one or more planes by actuating one or more bending sections of the bendable body 104. An example of a continuum robot is a snake-like catheter and/or endoscopic device, as described in applicant's previously published U.S. Pat. Nos. 9,144,370; 11,051,892; 11,103,992; 11,278,366; 11,007,641 and patent application publications US2019/0105468; US2021/0386972; US2021/0308423; and US 2022/0126060, which are incorporated by reference herein for all purposes.

FIG. 4 illustrates the system controller 128 executes software programs and controls the display controller 130 to display a navigation screen (e.g., a live view image 132) on the main display 118 and/or the secondary display 120. The display controller 130 may include a graphics processing unit (GPU) or a video display controller (VDC).

FIG. 5 illustrates components of the system controller 128 and/or the display controller 130. The system controller 128 and the display controller 130 can be configured separately. Alternatively, the system controller 128 and the display controller 102 can be configured as one device. In either case, the system controller 128 and the display controller 130 comprise substantially the same components. Specifically, the system controller 128 and display controller 130 may include a central processing unit (CPU 182) comprised of one or more processors (microprocessors), a random access memory (RAM 184) module, an input/output (I/O 186) interface, a read only memory (ROM 180), and data storage memory (e.g., a hard disk drive (HDD 188) or solid state drive (SSD)).

The ROM 180 and/or HDD 188 store the operating system (OS) software, and software programs necessary for executing the functions of the robotic catheter system 100 as a whole. The RAM 184 is used as a workspace memory. The CPU 182 executes the software programs developed in the RAM 184. The I/O 186 inputs, for example, positional information to the display controller 130, and outputs information for displaying the navigation screen to the one or more displays (main display 118 and/or secondary display 120). In the embodiments descried below, the navigation screen is a graphical user interface (GUI) generated by a software program but, it may also be generated by firmware, or a combination of software and firmware.

The system controller 128 may control the bendable body 104 based on any known kinematic algorithms applicable to continuum or snake-like catheter robots. For example, the system controller controls the bendable body 104 based on an algorithm known as follow the leader (FTL) algorithm. By applying the FTL algorithm, the most distal segment 156 of the steerable section 150 is actively controlled with forward kinematic values, while the middle segment 154 and the proximal segment 152 (following sections) of the bendable body 104 move at a first position in the same way as the distal section moved at the first position or a second position near the first position.

The display controller 130 acquires position information of the bendable body 104 from system controller 102. Alternatively, the display controller 130 may acquire the position information directly from the tip position detector 142. The bendable body 104 may be a single-use or limited-use catheter device. In other words, the bendable body 104 can be attachable to, and detachable from, the actuator unit 122 to be disposable.

During a procedure, the display controller 130 can generate and outputs a live-view image or other view(s) or a navigation screen to the main display 118 and/or the secondary display 120. This live-view image may be from a camera 140. This view can optionally be registered with a 3D model of a patient's anatomy (a branching structure) and the position information of at least a portion of the catheter (e.g., position of the catheter tip 158) by executing pre-programmed software routines. Upon completing navigation to a desired target, one or more end effector tools can be inserted through the access port 126 at the proximal end of the catheter, and such tools can be guided through the tool channel 168 of the catheter body to perform an intraluminal procedure from the distal end of the catheter.

The tool may be a medical tool such as an endoscope camera, forceps, a cryobiopsy probe, a needle or other biopsy or ablation tools. In one embodiment, the tool may be described as an operation tool or working tool. The working tool is inserted or removed through the working tool access port 126. In the embodiments below, an embodiment of using a bendable body to guide a tool to a target is explained. The tool may include an endoscope camera or an end effector tool, which can be guided through a bendable body under the same principles. In a procedure there is usually a planning procedure, a registration procedure, a targeting procedure, and an operation procedure.

For a medical procedure where the bendable medical device 104 will be used, medical images (e.g., from the CT scanner) are often pre-operatively provided to the robotic catheter system 100. With the robotic catheter system 100, a clinical user creates an anatomical computer model from the images, such as of the lung airways of patient 114. From chest images received from the CT scanner or PACS system, the clinical user can segment the lung airways for clinical treatments, such as a biopsy. After the robotic catheter system 100 generates a map of the lung airways, the user can also use the navigation software system to create a plan to access a lesion for the biopsy. The plan includes the target lesion and a trajectory (navigation path) through the airways to insert the distal section 150 of the bendable body 104.

According to one embodiment, either during insertion or retraction of the bendable body 104, the system controller 128 may control the linear translation stage 110 of the robotic platform 106 to move the bendable body 104 along the center line of a lumen (e.g., an airway) in a desired trajectory followed by active control of the bending segments. This is similar to known shaft guidance techniques used to control robotic guided catheters or endoscopes with the goal of forcing the flexible shaft of the sheath to keep to a desired trajectory. In one example, when using the robotic catheter system 100, the bendable body 104 is robotically controlled to advance through a lumen. The control may be through sensors, the external images, and/or images from the tip of the bendable body. Sensors may be included to measure the actuation force, insertion depth, the angulations of user-controlled steerable segments, etc., to obtain trajectory and other information. After a short advance in insertion or retraction distance, the shape of the bendable body 104 is corrected by adjusting (actuating) one or more of the bending segments in such a way that the new shape closely matches the desired trajectory. This process is repeated until a target area is reached. The same process can be applied when the bendable body is controlled to withdraw the bendable body 104 from the patient. This process is similar to the navigation process described in, e.g., US 2007/0135803, which is incorporated by reference herein for all purposes.

Additional details for driving a bendable body robot include the control methods for actuation, as described in applicant's previous patent application publications U.S. Pat. Nos. 11,051,892, 11,103,992, 11,278,366, \U.S. Pat. Nos. 11,007,641, 11,622,828 and U.S. patent application Ser. No. 18/816,580 filed 27 Aug. 2024 which are also incorporated by reference herein for all purposes.

### <Bending Modes>

The bendable body 104 can be actuated to bend each segment independently. While one mode of operation provides for the full control of each bendable segment independently, other modes can be provided that pre-define how the bendable medical device responds to movement commands. For example, in one bending mode, only bending the middle of the three segments provides a “chickenhead” motion. Another bending mode provides for all three bendable segments to bend uniformly.

To provide the multiple bending modes, a control algorithm can apply a different function for each of the bending sections to determine the amount and direction of motion that gets applied. This function will be based on the user (or system) supplied input motion, and can also consider any other number of arguments. Some of these optional arguments include the current control mode, the current/historical pose of each section of the catheter, and the location of the catheter within the anatomy.

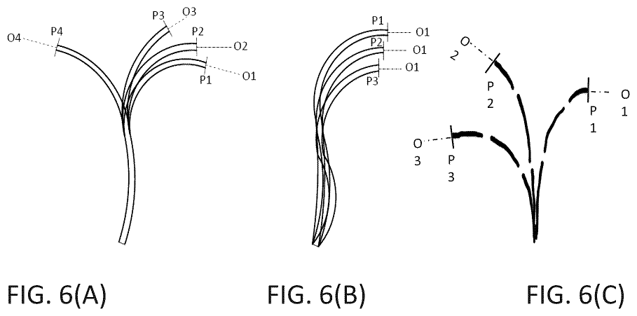

FIGS. 6(A)-6(C) show exemplary catheter tip manipulations by actuating one or more bending segments of the bendable body 104 in different bending modes. As illustrated in FIG. 6(A), manipulating only the most distal segment 156 of the steerable section (tip bending mode) changes the position and orientation of the catheter tip 158. On the other hand, manipulating one or more bending segments (152 or 154) other than the most distal segment affects only the position of catheter tip 158, but does not affect the orientation of the catheter tip. In FIG. 6(A), actuation of distal segment 155 changes the catheter tip from a position P1 having orientation O1, to a position P2 having orientation O2, to position P3 having orientation O3, to position P4 having orientation O4, etc. In FIG. 6(B) actuation of the middle segment 154 changes the position of catheter tip 158 from a position P1 having orientation O1 to a position P2 and position P3 having the same orientation O1. This targeting mode is also described as a Chicken Head mode due to the way the tip can move around the target site like a chicken searching for grain.

Here, it should be appreciated by those skilled in the art that exemplary catheter tip manipulations shown in FIGS. 6(A) and 6(B) can be performed during catheter navigation (i.e., while inserting the catheter through tortuous anatomies). In the present disclosure, the exemplary catheter tip manipulations shown in FIGS. 6(A) and 6(B) apply namely to the targeting mode. In some examples, this mode is applied after the catheter tip has been navigated to a predetermined distance (a targeting distance) from the target. In FIG. 6(C) the bending is distributed through the different bending sections. The actuation of all three segments changes the catheter tip from a position P1 having orientation O1, to a position P2 having orientation O2, to position P3 having orientation O3, etc. While this mode may not be useful in navigating all tightly turning anatomy, as can be seen by comparing FIG. 6(A) with FIG. 6(C), it can be particularly important for tool ingress where the tool may be too long to bend as tightly as was used to navigate to the target site.

For each of these, modes, input data is used to move the catheter segments. Input data may come from a user, an automated system (see, for example, PCT/US24/37935 or PCT/US24/37924 filed 12 Jul. 2024), or a combination thereof. For example, input data includes a selection of operation mode, a target bend from a user via a hand controller or GUI. The target bend can be the bend angle, the bend direction, or a combination. It includes both magnitude and direction. In some embodiments, the function applied to the magnitude and the function applied to the direction can be the same or it can be different.

Alternatively, or additionally the input date may be a data representation of bend angle, which is a wire position or other kinematic representation that is an alternative expression of the bend angle and/or direction. This data representation may be a translation of the user-defined target bend translated into information the controller uses to control the drive wires.

The target bend may be the bend angle (or direction) for the final target (e.g., bending to the region of interest). In other embodiments, the target bend is a change in bend angle (or direction).

Other input instruction may include the current or prior bend angle of each segment and/or data representation of the current or prior bend angle. This information can be obtained, for example, from a table that stores pose information for a procedure.

In Tip Bending mode, the controller is configured to move the at least one distal drive towards the target bend, or the data representation of bend angle, based on input data. This bend is to the bend angle and/or direction resulting from the prior bend angle and/or prior direction.

In Targeting mode, the controller is configured to move the at least one proximal drive wire towards the target bend or the data representation based on input data. The proximal drive wire is moved in the case of the bendable body having two bending segments. However, when the bendable body has three or more bending segments, the controller is configured to move only an at least one drive wire to bend the bending segment proximal to the distal bending segment towards the target bend or the data representation of bend angle based on input data.

Distributed bending mode. In this mode, the controller is configured to move the at least one proximal drive wire and the at least one distal drive wire simultaneously. This may accomplish bending the proximal bending segment at an angle less than the bend angle and to bend the distal bending segment to the bend angle. These can be in any ratios, the proximal doesn't necessarily have to be less than distal, and neither technically have to utilize the “full” bend angle/direction.

### <Distributed Bending and the Distribution Function>

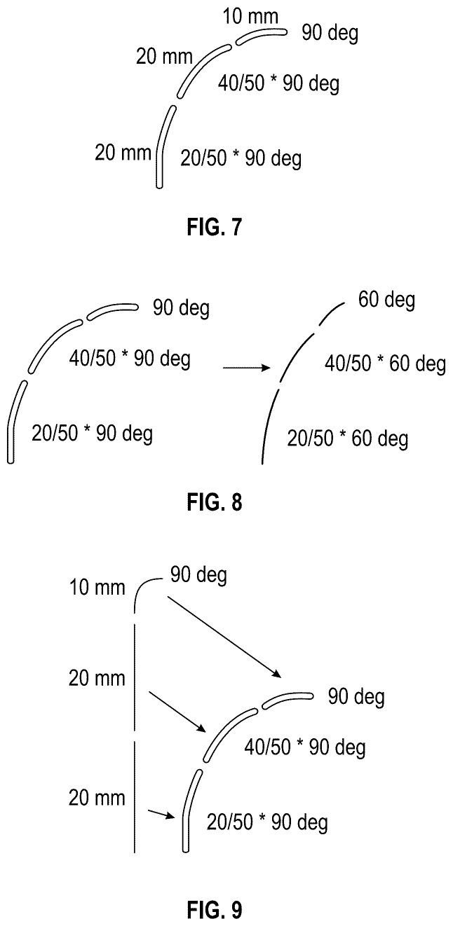

The most basic function for distributed bending is a simple proportional amount of the input value. For example, to create a uniform curvature using all bending sections, the amount of motion applied to each section will match the percentage of the position the end effector is along the entirety of the combined bending length. This can be seen in FIG. 7. Looking at the exemplary end of the bendable body having three bending segments, the proximal bending segment 152 is seen with a length of 20 mm. The middle segment 154 and distal segment 156 are shown as having a total length of 30 mm for a total bendable length of 50 mm. Thus, for a 90 degree turn of the bendable body in FIG. 7, the proximal bending segment 152 has an angle of 20 mm/50 mm*90 deg.=36 deg., which is the percentage of the position of the end effector along the combined bending length. The middle bending segment 154 will have an amount of motion applied to match the equation 40 mm/50 mm*90 deg.=72 deg. since this segment is 40 mm from the base position and the total length is 50 mm. The distal segment 156 will have the full 90 deg. bend.

While FIG. 1 is shown for a three-section bendable body having the lengths of 20, 20, and 10 mm, other embodiments can have two, four, or more sections that have the same or different lengths. The important consideration for this most basic function of distributed bending is that the proportional amount of the input value remains constant. For example, a four-section robot with each section having a 20 mm length, with a target bend having an angle of 60 degrees, would have a distal segment bending at 15 deg., the middle segments bending at 30 and 45 deg., and the proximal segment bending at the listed 60 deg.

Similarly, Tip Bending and Chickenhead motion (as shown in FIGS. 6(A) and 6(B)) fall under this algorithm if this control mode is included in the function. In Tip Bending mode, the tip receives 100% of the motion while all other sections receive 0% (FIG. 6(A)). Likewise, in Targeting/Chickenhead mode (FIG. 6(B)), the middle segment receives 100% of the motion while all other sections received 0%.

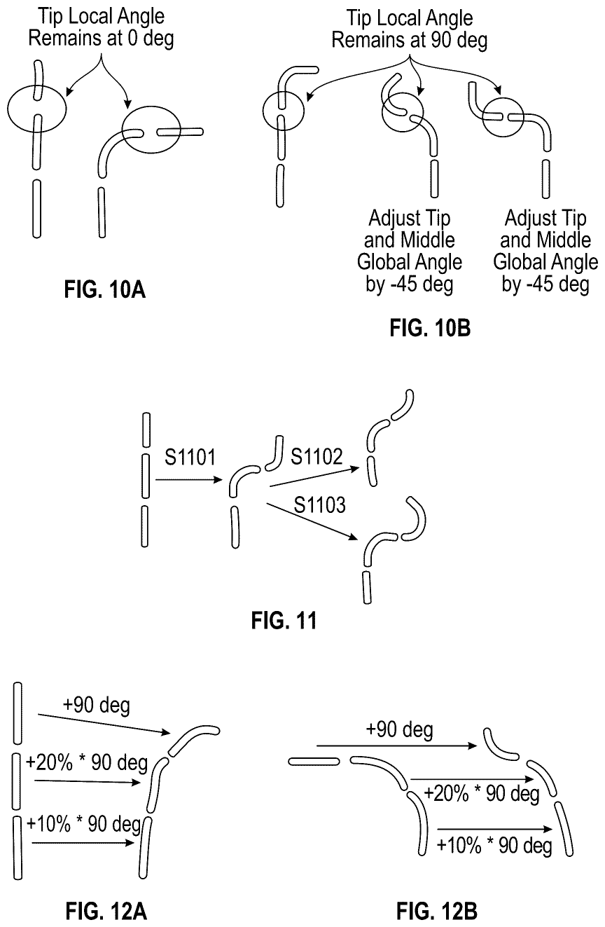

However, there are certain functions that are not possible with targeting and chicken head motion. For example, there are certain tools that are very stiff at the distal portion. This can restrict the amount of local bending that the tip can achieve. (Local bending is the relative pose between the tip and middle segments). In this scenario, if the user wanted to bend the catheter in a direction that the tool is prohibiting, the motion can be applied to both the tip and middle segment equally in a distributed bending mode (FIG. 10(B)). This will therefore change the tip orientation without changing the local pose.

This simple ratio distribution can also be used to implement functions that are not currently possible with other systems.

The table below is a simple example of the aforementioned examples with a catheter having three bendable sections of 10, 20, and 20 mm.

TABLE 1
Movement Modes for 10 mm/20 mm/20 mm Catheter

| | Distal % | Middle % | Proximal % |
|---|---|---|---|
| Tip Bending Mode | 100 | 0 | 0 |
| Chickenhead Mode | 0 | 100 | 0 |
| Targeting or Orientation Mode | 100 | 100 | 0 |
| (Change Tip Orientation without | | | |
| Changing Local Pose) | | | |
| Distributed Bending Mode | 100 | 80 | 40 |

This distribution function does not merely have to modify the amount of motion; it can also affect the direction. This will be useful for Chickenhead motion. The desired effect of chickenhead motion is to translate the tip position without changing the orientation. It achieves this by bending the middle segment. However, due to the physics of the catheter design, the orientation of the tip will change slightly as the middle segment moves. As shown in FIG. 11, this sort of motion can be counteracted by applying a bending to the tip to offset the resultant motion from the middle segment bending. The direction of this would typically be in the opposite direction of the middle segment motion.

The amount of bending to be applied, however, would depend on the local pose of the tip. A consequence of bending the middle segment is that the local pose of the tip section will change. As mentioned earlier, the local pose is the relative pose between the tip section and the middle segment. One physical phenomenon of the three-section catheter is that output ratio is inversely proportional to its local pose. In other words, the same drive wire positions will produce a lower output angle as the local pose angle increases. Therefore, the degree to which the tip orientation will change from chickenhead motion will vary based on how its local pose changes. So, the function to counteract this unintended tip orientation change will have to use the local pose in its calculations.

Finally, if the same basic ratio distribution is applied to the catheter motion from the start of control, then the resultant pose of all sections at any point during the operation is in the same ratio distribution.

In FIG. 8, a catheter with the uniform curvature distribution (at 90 degrees) gets applied a uniform curvature distribution motion of 30 degrees. The resultant pose is still a uniform curvature distribution (at 60 degrees).

However, the pose of the catheter before applying any sort of distributed bending motion is not required to be distributed in the same ratio at the motion. In other words, any sort of distributed bending motion algorithm can be applied to any sort of catheter pose (See FIG. 12). They are independent of each other.

One significant implementation of this is during simple navigation. There are scenarios where the catheter tip bending alone is not sufficient to orient the tip in the desired direction because it reaches its physical limits (the local pose is too high). In prior catheters, when this happens, the stage is driven forward and the FTL algorithm moves the middle segment towards the tip direction. This will reduce the local pose of the tip section, permitting it the ability to continue aiming in the direction it was not able to reach previously. (i.e., increase the local pose further).

This utility, of slightly moving the middle segment in the direction that the tip is trying to aim, can be replicated using this distributed bending. By applying a partial motion to the middle segment, and optionally the proximal segment, to shift the entire bending portion in the direction that the tip is bending, the utility is achieved. This will reduce the amount of local pose increase that occurs during bending, permitting the tip to bend further than it otherwise would be able to bend. Again, the amount of bending that gets applied to the middle/proximal segment could be a function of the local pose. The larger the local pose, the more benefit is obtained from directing the middle segment to move by a larger amount. This could also improve FTL efficiency, as the local pose will be less than it would be without distributed bending mode.

Finally, the functions can also be entirely user define, or have certain parameters that are user defined, and can be adjusted at any point (manually or automatically). One scenario where it would be beneficial to have them change “automatically” could be implemented for “cascading bending”.

In another type of distributed bending mode, the bendable device is initially bent with uniform curvature as discussed above. However, when a specified limit has been reached, the controller stops the motion of the proximal most section but continues the motion in the more distal segments. If there are more than 2 bending sections, this cycle can repeat of all sections.

In yet another example, where the Tip Bending mode is initially used, this mode is used until a local pose limit has been reached. When this occurs, any further bending will then also be applied to both the distal segment and the prior segment (the middle segment in a 3-section catheter). If the catheter has more than 2 sections, this sequence can repeat.

The bending does not have to be limited to a single section at a time, it can be distributed through a manner explained in the previous section, but still follow the same paradigm of applying the motion in the other sections once a limit has been reached.

In some embodiments, the constant curvature mode as described in WO/2023/164047 to Takagi may be used. This publication describes a bendable body that is bent by a controller that is configured to switch between first control for bending the first bending portion and second control for bending the first and second bending portions, so they have constant curvature. WO/2023/164047 is hereby incorporated by reference in its entirety.

<Transitioning into a Derived Pose>

The user can transition into any distributed pose from any other pose. When this happens, the pose of all sections will be adjusted to match the desired distribution based on the tip's pose. Optionally, the use can only apply this transition to specific sections.

The user can also have the option to revert back to the prior pose from a distributed pose (or, they can select which sections should be reverted, while maintaining others). Finally, the user can change the distributed bending ratio (i.e. enter chickenhead mode) from this distributed pose mode. They would have the option to also change the current pose to match the new distribution, or only have the distribution be applied during bending.

It is also possible for this transition to be applied at a gradual basis, and the user can stop it at any point during the transition if a more desirable pose calls for it.

When transitioning into a distributed pose, the orientation of the tip should remain the same, but the position in space would change. This position change might be able to be accounted for, either manually or automatically, by moving the stage position or bending other sections accordingly.

Also, the function to determine the pose of each section does not have to be based on the tip pose, it can be based on the pose of any section and/or other information such as target location, etc.

### <Additional Distribution Functions>

In some embodiments, the distribution functions of bending through more than one bending section.

One function provides for improved maintenance of the local pose is the Orientation Mode, shown in FIGS. 10(A) and 10(B). The local angle is maintained while still changing the orientation. For a three-section robot, the configuration is the distal segment is at 100%, the middle segment is at 100%, and the proximal segment is at 0%. As shown in FIG. 10(A), the distal segment is straight, relative to the middle segment. When a bend command of 90 deg. is sent, the result is that the distal and middle segments both aiming towards 90 deg and the distal segment is still straight relative to the middle. In FIG. 10(B), the scenario is that the distal segment is at +90 deg, relative to the middle segment. When a bend command of −45 deg. is sent, the result is that the distal and middle segments both change aiming direction by −45 deg. The distal segment is still bent +90 deg. relative to the middle segment.

Another function of distributed bending relates to Chickenhead mode. In standard Chickenhead mode, the middle segment is bent while the distal and proximal are not. However, there is often some crosstalk between the distal and middle segments and the true final orientation is not accurate. As shown in the example of FIG. 11, all three sections are straight in the three-section robot. A command to bend 90 degrees is sent [S1101] to provide a middle segment bent by 90 deg. However, the true output shape of the standard Chickenhead mode is shown through the bending of where the tip of the distal segment is pulled slightly towards the middle segment bending direction. The tip local angle is reduced, and the tip is no longer aiming straight ahead. With a distributed function, shown in step [S1103], the distal segment is bend in the opposite direction of the middle segment bend ([S1101]) to counteract the pull of the middle segment bending on the distal segment. This increases the local angle at the tip and the output direction remains straight ahead.

In yet another example of a distributed function, the middle and/or proximal segment can be “nudged” towards the tip. The settings of an example of this mode are:

TABLE 2
Movement Mode for 10 mm/20 mm/20 mm Catheter

| | Distal % | Middle % | Proximal % |
|---|---|---|---|
| Nudge | 100 | 20 | 10 |

This can be used to reduce the local angle to increase output. As shown in FIG. 12(A), all sections are straight. When a command to bend +90 deg is sent, the distal segment bends 90 deg., the middle segment bends 20%, or 18 deg. and the proximal segment bends 10%, or 9 deg. All the bending occurs in the direction of the tip bending. This can be seen differently in FIG. 12(B) where all sections are angled left, with varying angles at the start of the movement. When a +90-deg. bend command is sent in this case, the distal segment bends by 90 deg., the middle segment bends by 18 deg, and the proximal segment bends by 9 degrees, all in the direction of tip bending. However, the overall shape of this final curve is as if the tip of the robot moves as directed and the other sections are nudged in the direction of the bend to provide a shape that may be particularly useful when navigating and working within a lumen.

In other embodiments, the amount of the nudge may be greater (e.g., 30% and 15%) or less (e.g., 10% and 5%), or may be at a different proportion (e.g., 30% and 10%).

### <Tool Passage and the User Interface>

Because the structure of the airway can be torturous, and the size of the airways decrease as the branches move away from the trachea, navigation to a peripheral area can be difficult. Once the airway is too small for the passage of the bendable body, navigation through the airway cannot happen. But even before this size constraint limits a procedure, a torturous pathway with, potential, multiple bends and bends having a tight radius of the bendable body as it approaches a region of interest may be created. While the bendable body as described herein may have sufficient flexibility and turn radius to reach a region of interest, there is still a need to provide one or more tools at the region of interest. Many tools do not have a flexibility similar to the bendable body or even a camera inserted into the bendable body for navigation. Thus, the tool could have significantly more difficulty passing thought the bendable body and particularly through a torturous path to the region of interest that a bendable body with a camera in the tool channel.

The distributed bending mode can be used to improve the insertion of some tools. An advantage of the distributed bending mode is that it creates a gentler curve to pass a tool through, which allows the rigid tip of the tool to pass. The modes could be controlled by a button or switch, which toggles between the two states and thus it can be used by the clinician any time a tool is inserted into the catheter or whenever a gentler curve is advised.

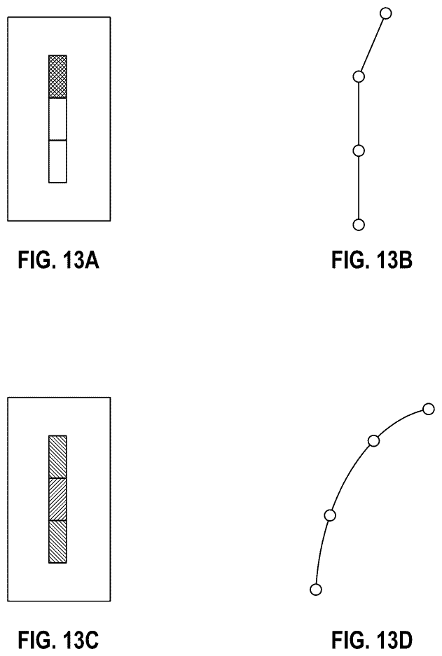

To aid the user in choosing the appropriate mode as well as inform the user about the differences and/or uses of the different modes, the system can provide a visualization on the display to show the user what the current mode. For example, the display of FIG. 13(A) graphically shows the tip bending mode while the display of FIG. 13(C) shows the distributed bending mode, where the number of bending segments that are bending is distinguished from the number of bending segments that are not being bent. FIGS. 13(B) and 13(D) similarly show the tip bending mode and the distributed bending mode, respectively, but where-a simple line or arc is provided to indicate the segments of the bending section that are bent.

### <Workflow>

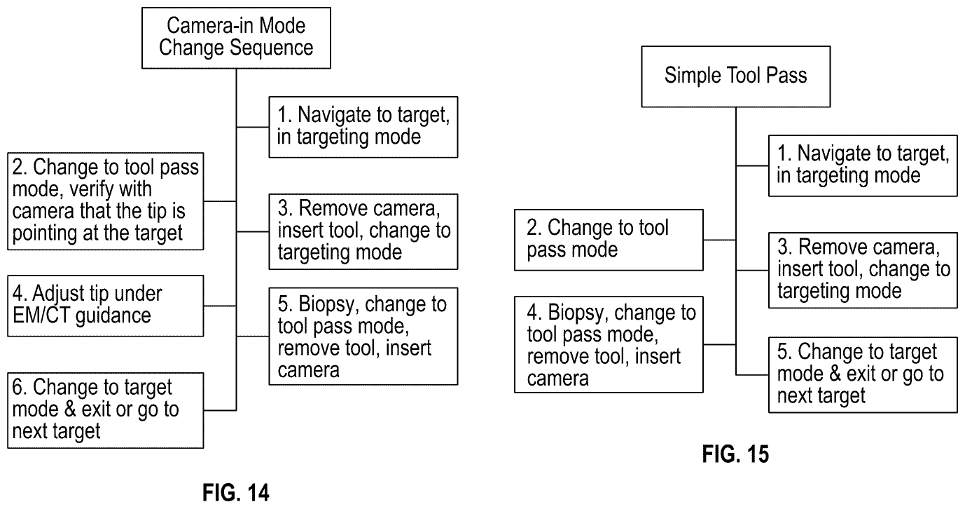

This tool insertion mode could be enabled in the workflow shown in FIG. 14. In this workflow, the user navigates the bendable body to a target in tip bending mode (also described as targeting mode). Once near the target or other region of interest, the mode is changed to distributed bending mode. The position of the tip relative to the region of interest is verified with the camera. The camera is removed and a tool, such as a biopsy needle, is inserted into the catheter. For fine adjustments, the mode is changed to tip bending mode. The position then can be adjusted under EM or CT guidance. Next, the biopsy, drug delivery, ablation, or other surgical intervention is provided. Once complete, the mode is changed one again to distributed bending mode and the tool is removed. The camera is then re-inserted. The mode is changed to tip bending mode and the catheter is either removed from the patient or moved to another region of interest.

FIG. 15 provides a simplified tool pass workflow. As previously, the user navigates the bendable body to a target in tip bending mode. Once near the target or other region of interest, the mode is changed to distributed bending mode. The camera is removed, and a tool is inserted into the catheter. The mode is changed to tip bending mode. A biopsy, drug delivery, ablation, or other surgical intervention is completed, and the mode is then changed to distributed bending mode and the tool is removed. The mode is changed to tip bending mode and the catheter is either removed from the patient or moved to another region of interest.

The workflow for changing in and out of distributed bending mode may be one of two methods. The first is where the switching is activated by the user, where an indicator of the need and/or usefulness of changing mode is shown as applicable. The second is where the switching is activated by the controller based on detected information such as when the catheter reaches within a distance D to a ROI or when the bend status is found to be above a threshold T.

### <Tool Passability Visualization>

In some embodiments, system feedback from the system is provided to indicate to the user that the tool may be difficult or impossible to pass through the tool channel in the current mode and/or to prompt the user to switch between tip bending mode and distributed bending mode.

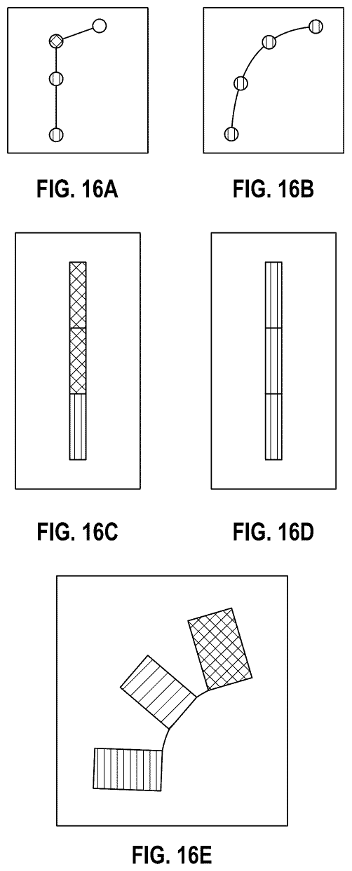

For example, FIG. 16(A) provides a visualization of the catheter and the three bending modes, where the bending segments (or the juncture between the segments) is shown as passable or not passable. Segment/joint graphical change concept is the use of color or other graphical means to convey if tools can pass a particular juncture. For example, a node or section is colored green if the tools can pass. Other colors or indicates such as yellow or orange as catheter curvature could pose difficulty, and then red for the segments where the tool cannot pass. Alternatively, other colors or indicator scheme (e.g., icons such as check marks, X or other graphics, a sound where a change in the sound indicates passability/lack of passability) could be used. In this example, the red dot indicates that the tool cannot pass through the bendable body at this segment. In contrast, FIG. 16(B) shows the catheter in distributed bending mode and all nodes are green, indicating that the tool can pass through the catheter. Similarly, FIG. 16(C) is another way of showing that a tool cannot pass (in this example, the tool cannot pass through the two most distal segments, where in FIG. 16(D), the tool can pass through the entire bendable body. The passability may be shown with the curvature of the bendable body (FIGS. 16(A) and 16(B) or a simplified version showing only the segments (FIGS. 16(C) and 16(D).

In another embodiment, shown in FIG. 16(E), the three segments of the catheter are shown, each with a color indicating the passability of a specified tool. In this example, the most proximal segment is green, indicting the tool will pass. The second segment is yellow, indicating a possible but potentially difficult passability. The addition of an intermediate indicator could be important since the lung is pliable and, while the curvature may be insufficient to pass, by pushing the tool through, the lung will deform sufficiently to pass the catheter. However, the user may have varying constraints as to how much force would be advisable for any specific patient and/or procedure. The third segment of FIG. 16(E) is red, indicating that the tool will not pass through the tool channel of this segment in the current bending configuration. In some embodiments, the graphic similar to that shown in FIG. 16 (A), (B), or (E) will be updated continuously or periodically as the bendable body is moved through the lumen and the curvature of each of the bendable segments changes, thereby changing the passability.

In one example, the bendable body has three bendable segments, and the distal tip has at least an 8-, 7-, 6-, or 5-mm degree of curvature. The tool channel is between 2.5 and 1.9 mm in diameter (e.g., 2.1 mm) through the bendable body.

### <Notifications>

In some embodiments, the system is configured to notify the user that a switch to distributed bending mode may be advised due to the curvature of the catheter and/or the size and configuration of the tool being used. Alternatively, or in addition, the system may be configured to notify the user that a mode change is indicated when the camera or other tool is removed or inserted into the catheter and the mode is not changed for this process.

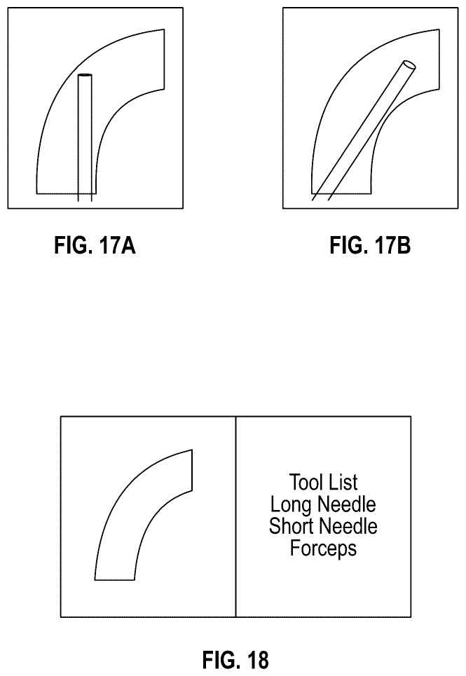

These notifications may be done in any format, such as a pop-up box on the screen. In some embodiments, a display of the current issue, either an image or a simplified depiction, can be used. For example, the display shown in FIG. 17(A) showing the current curvature and the rigid length of a tool which is not passable in the shown configuration. In contrast, FIG. 17(B) shows the bendable body in distributed bending mode where the tool is shown as passable.

In other embodiments, the system will notify the user when the system senses proximity to a lesion or other region of interest (ROI). When a certain proximity (distance D) to the ROI is reached, the system sends a recommendation to the display for a mode switch to biopsy/distributed bending mode. This indicates an end to the navigation step and begins the assessment or treatment step. Thus, a user may be navigating through the lung towards a lesion or other ROI. When the distal tip of the steerable distal section of the bendable body reaches within a distance D of the ROI, where D is, for example, 30 mm or less, 20 mm or less, 18 mm or less, 16 mm or less, 14 mm or less, 12 mm or less, 10 mm or less, 8 mm or less, 6 mm or less, or 5 mm or less from the ROI (measured either as from the center of the ROI or from the closest point), the system will recognize the relative location of the tip of the medical instrument and the ROI and notify the user by a visual or sound. For example, the GUI may provide a statement on the display that reads “switching modes is recommended,” “ready for biopsy mode” or any other informational communication to the user.

For this notification to occur, the user or the system must have information as to the location of the lesion or other ROI. This may be obtained from a Planning step—either directly prior to the navigation or earlier and may involve visualization from an external imaging source such as CT or fluoroscopy where the ROI is marked by the user or was previously indicated.

The system recognizes the relative location of the tip to the ROI (or to other locations within the lung or other location through one of several ways, including an EM sensor on or near the distal tip of the steerable distal section. The system may also recognize the relative location through an image or images from the camera in combination with an algorithm, such as a machine learning algorithm. In other embodiments, the user can input this information.

In some embodiments, the system will notify the user when a bend status is above a threshold, T. In such instances, the bend radius of the distal end of the bendable device may be at or near the maximum bend radius for the device or at or near the maximum bend radius through which a tool can pass, and the bend threshold is set at this maximum bend radius or some fraction thereof. In some embodiments, if the force on one or more of the drive wires is above a certain value, this indicates that it may be outside of normal operation and affecting the bending and control of the bendable body, so the bend threshold T can be set based on the drive wire force.

The bend status may be monitored or measured by a shape sensor at or near the distal end of the bendable body or at any point in the bendable area of the bendable body. In some embodiments, the bend status may be monitored or measured by a wire force measurement. Sensors that sense when a tool is inserted into or removed from the bendable body are also used, as well as sensors that indicate where the bendable body is, for example, in relation to the navigation path. This sensor (or sensors) sends information to the controller which may then activate the switch to change between a movement mode and the distributed bending mode.

### <Proximity Switching & Force Switching>

The system can also be configured to automatically switch to the distributed bending mode upon detection of camera or tool removal. For this embodiment, a sensor, either at the distal tip of the bendable body or more proximal to the insertion point can be used to inform the system that a camera or tool has been removed. This automatic switching is advantageous in that it removes the steps of switching back and forth allows for easier tool insertion. The system can also automatically switch back to tip bending mode once the biopsy or other procedure is complete or once the camera is re-inserted after the procedure.

The system can also be configured to automatically switch to the distributed bending mode upon the bendable body that the steerable distal section has reached a location within a distance D of a region of interest, or when the bend status is above a bend threshold T. In some embodiments, the distance D is set by a user prior to the procedure. The bend threshold T may be set by a user or may be automatic, based on parameters defined by the bendable body or by the tool.

### <Passable Tool List>

In some embodiments, the system provides information to the user about the tools that are passable through the bendable body. The system can display a prompt to signal to the user which tools can be passed through the tool channel of the bendable body.

As shown in FIG. 18, the current curvature is displayed (either visually as provided in this figure, or numerically, etc.) A list of passable tools is also displayed list concept is a dialog box which, given the current curvature, lists the tools which will pass through. That is, as the curve becomes more tight, fewer and fewer tools will be listed.

The screen may show currently passable tools and also list the tools that are passable after changing the mode from tip bending mode to biopsy mode. FIG. 18 is an illustration of a screen showing the curved distal end of the bendable body and a list of the three tools that can pass. As the bendable body is moved further into the lumen and as the pathway becomes more tortious, the number of tools on the list would decrease to include only the tools that could pass. In some embodiments, the list provided is a listing of the tools that can pass through the bendable body in its current configuration. In other embodiments, a second list is also shown (or the second list replaces the first) where the second list provides a listing of the tools that could pass through the bendable body in the distributed bending mode.

The list of passable tools may be selected from a library of tools that can be used with the system as described herein. The diameter and rigid length of the tools can be obtained from the tool manufacture, may be inserted by the user, or may be provided as a library by the system. This information is then used, along with the diameter of the tool channel and the specific geometry of the various bendable segments.

### <Computer Control>

Embodiment(s) of the present disclosure can be realized by computer system 400 or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer system may comprise one or more processors (e.g., central processing unit (CPU) 410, micro processing unit (MPU)) and may include a network of separate computers or separate processors to read out and execute the computer executable instructions. The computer-executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like. An I/O interface can be used to provide communication interfaces to input and output devices, which may include a keyboard, a display, a mouse, a touch screen, touchless interface (e.g., a gesture recognition device) a printing device, a light pen, an optical storage device, a scanner, a microphone, a camera, a drive, communication cable and a network (either wired or wireless).

The present disclosure and/or one or more components of devices, systems, and storage mediums, and/or methods, thereof also may be used in conjunction with continuum robot devices, systems, methods, and/or storage mediums and/or with endoscope devices, systems, methods, and/or storage mediums. Such continuum robot devices, systems, methods, and/or storage mediums are disclosed in at least: U.S. Pat. Nos. 10,687,694; 11,007,641; 11,051,892; 11,096,552; 11278366; 11,504,501; 11,559,190; 11,571,268; and 11,622,828; U.S. Pat. Publications 2020/0375665; 2021/0121051; 2021/0121162; 2021/0259521; 2021/0259790; 2021/0259794; 2021/0260339; 2021/0260767; 2021/0308423; 2021/0362323; 2021/0369085; 2021/0369355; 2021/0369366; 2021/0386972; 2021/229277; 2022/0016394; 2022/0039635; 2022/0039784; 2022/0040450; 2022/0126060; 2022/0202273; 2022/0202277; 2022/0202500; 2022/0202501; 2022/0202502; 2022/0203071; 2023/0016761; 2023/0201522; 2023/0137954; and 2023/0255442 as well as international Publication numbers WO2021/142272; WO2022/031995; WO2022/032162; WO/2023/154352; WO/2023/154825; WO/2023/154931; WO/2023/150162; WO/2023/154713; WO/2023/154246; and WO/2023/154931, the disclosure of each of which are incorporated by reference herein in their entirety. Any of the features of the present disclosure may be used in combination with any of the features as discussed in the patents and applications above.

### Definitions

Throughout the figures, where possible, the same reference numerals and characters, unless otherwise stated, are used to denote like features, elements, components or portions of the illustrated embodiments. In addition, while the subject disclosure is described in detail with reference to the enclosed figures, it is done so in connection with illustrative exemplary embodiments. It is intended that changes and modifications can be made to the described exemplary embodiments without departing from the true scope of the subject disclosure as defined by the appended claims. Although the drawings represent some possible configurations and approaches, the drawings are not necessarily to scale and certain features may be exaggerated, removed, or partially sectioned to better illustrate and explain certain aspects of the present disclosure. The descriptions set forth herein are not intended to be exhaustive or otherwise limit or restrict the claims to the precise forms and configurations shown in the drawings and disclosed in the following detailed description.

It should be understood that if an element or part is referred herein as being “on”, “against”, “connected to”, or “coupled to” another element or part, then it can be directly on, against, connected or coupled to the other element or part, or intervening elements or parts may be present. In contrast, if an element is referred to as being “directly on”, “directly connected to”, or “directly coupled to” another element or part, then there are no intervening elements or parts present. When used, term “and/or”, includes any and all combinations of one or more of the associated listed items, if so provided.

Spatially relative terms, such as “under” “beneath”, “below”, “lower”, “above”, “upper”, “proximal”, “distal”, and the like, may be used herein for ease of description to describe one element or feature's relationship to another element(s) or feature(s) as illustrated in the various figures. It should be understood, however, that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, a relative spatial term such as “below” can encompass both an orientation of above and below. The device may be otherwise oriented (rotated 90 degrees or at other orientations) and the spatially relative descriptors used herein are to be interpreted accordingly. Similarly, the relative spatial terms “proximal” and “distal” may also be interchangeable, where applicable.

The term “about,” as used herein means, for example, within 10%, within 5%, or less. In some embodiments, the term “about” may mean within measurement error.

The terms first, second, third, etc. may be used herein to describe various elements, components, regions, parts and/or sections. It should be understood that these elements, components, regions, parts and/or sections should not be limited by these terms. These terms have been used only to distinguish one element, component, region, part, or section from another region, part, or section. Thus, a first element, component, region, part, or section discussed below could be termed a second element, component, region, part, or section without departing from the teachings herein.

The terminology used herein is for the purpose of describing particular embodiments only and is not intended to be limiting. The use of the terms “a” and “an” and “the” and similar referents in the context of describing the disclosure (especially in the context of the following claims) are to be construed to cover both the singular and the plural, unless otherwise indicated herein or clearly contradicted by context. The terms “comprising,” “having,” “includes”, “including,” and “containing” are to be construed as open-ended terms (i.e., meaning “including, but not limited to,”) unless otherwise noted. Specifically, these terms, when used in the present specification, specify the presence of stated features, integers, steps, operations, elements, and/or components, but do not preclude the presence or addition of one or more other features, integers, steps, operations, elements, components, and/or groups thereof not explicitly stated. Recitation of ranges of values herein are merely intended to serve as a shorthand method of referring individually to each separate value falling within the range, unless otherwise indicated herein, and each separate value is incorporated into the specification as if it were individually recited herein. For example, if the range 10-15 is disclosed, then 11, 12, 13, and 14 are also disclosed. All methods described herein can be performed in any suitable order unless otherwise indicated herein or otherwise clearly contradicted by context. The use of any and all examples, or exemplary language (e.g., “such as”) provided herein, is intended merely to better illuminate the disclosure and does not pose a limitation on the scope of the disclosure unless otherwise claimed. No language in the specification should be construed as indicating any non-claimed element as essential to the practice of the disclosure.

The present disclosure generally relates to medical devices, and it exemplifies embodiments of a bendable body having a steerable distal section. In some embodiments, this can be a catheter or may contain an optical probe which may be applicable to an imaging apparatus (e.g., an endoscope). The imaging apparatus may image using a miniature camera based on chip-on-tip (COT) technology or may provide some other form of imaging such as spectrally encoded endoscopy (SEE) imaging technology (see, e.g., U.S. Pat. Nos. 10,288,868 and 10,261,223). In some embodiments, the imaging apparatus may include an optical coherence tomographic (OCT) apparatus, a spectroscopy apparatus, or a combination of such apparatuses (e.g., a multi-modality imaging probe).

The embodiments of the bendable medical device and portions thereof are described in terms of their position/orientation in a three-dimensional space. As used herein, the term “position” refers to the location of an object or a portion of an object in the three-dimensional space (e.g., three degrees of translational freedom along Cartesian X, Y, Z coordinates); the term “orientation” refers to the rotational placement of an object or a portion of an object (three degrees of rotational freedom—e.g., roll, pitch, and yaw); the term “posture” refers to the position of an object or a portion of an object in at least one degree of translational freedom and to the orientation of that object or portion of object in at least one degree of rotational freedom (up to a total six degrees of freedom); the term “shape” refers to a set of posture, positions, and/or orientations measured along the elongated body of the object. As it is known in the field of medical devices, the terms “proximal” and “distal” are used with reference to the manipulation of an end of an instrument extending from the user to a surgical or diagnostic site. In this regard, the term “proximal” refers to the portion of the instrument closer to the user, and the term “distal” refers to the portion of the instrument further away from the user and closer to a surgical or diagnostic site.

As used herein the term “catheter” generally refers to a flexible and thin tubular instrument made of medical grade material designed to be inserted through a narrow opening into a bodily lumen (e.g., a vessel) to perform a broad range of medical functions. The catheter may be solely an imaging apparatus, or it may comprise tools for use in therapeutic or diagnostic procedures. The more specific term “optical catheter” refers to a medical instrument comprising an elongated bundle of one or more flexible light conducting fibers disposed inside a protective sheath made of medical grade material and having an optical imaging function. A particular example of an optical catheter is fiber optic catheter which comprises a sheath, a coil, a protector and an optical probe. In some applications a catheter may include a “guide catheter” which functions similarly to a sheath.

As used herein the term “endoscope” refers to a rigid or flexible medical instrument which uses light guided by an optical probe to look inside a body cavity or organ. A medical procedure, in which an endoscope is inserted through a natural opening, is called an endoscopy. Specialized endoscopes are generally named for how or where the endoscope is intended to be used, such as the bronchoscope (mouth), sigmoidoscope (rectum), cystoscope (bladder), nephoscope (kidney), bronchoscope (bronchi), laryngoscope (larynx), otoscope (ear), arthroscope (joint), laparoscope (abdomen), and gastrointestinal endoscopes.

It will be appreciated that the methods and compositions of the instant disclosure can be incorporated in the form of a variety of embodiments, only a few of which are disclosed herein. Variations of those embodiments may become apparent to those of ordinary skill in the art upon reading the foregoing description. Skilled artisans are expected and understood to employ such variations as appropriate, and the present disclosure is intended to be practiced otherwise than as specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

While the present disclosure has been described with reference to exemplary embodiments, it is to be understood that the disclosure is not limited to the disclosed exemplary embodiments. The scope of the following claims is to be accorded the broadest interpretation so as to encompass all such modifications and equivalent structures and functions.

1. A medical system comprising:
a bendable body having a steerable distal section comprising a plurality of steerable segments and a tool channel configured to hold a medical tool or a camera that extends through the steerable distal section;
a display device; and
a controller configured to:
control the steerable distal section in a first bending mode and in a distributed bending mode and
switch between the first bending mode and the distributed bending mode,
wherein the switching function is configured to perform at least one of:
(a) be activated by a user, and wherein the controller is further configured to display on the display device an indicator of a current mode and an indicator that the mode should be changed into or out of the distributed bending mode,
(b) be activated by a user, and wherein the controller is further configured to display a notification on the display device that the steerable distal section has reached a location within a distance D of a region of interest, or when a bend status is above a bend threshold T,
(c) be activated when the controller detects a camera or a biopsy tool being removed from the bendable body, and/or
(d) be activated when controller receives information that the steerable distal section has reached a location within a distance D of a region of interest, or when the bend status is above a bend threshold T.

2. The medical system of claim 1, wherein the controller is further configured to assesses whether a tool can pass through the steerable distal section and wherein the controller is further configured to display an indication of whether a tool can pass through the tool channel.

3. The medical system of claim 2, wherein the indication of whether a tool can pass through the tool channel is a color-coded representation of the steerable distal section or the representation of the steerable distal section is straight or bent to indicate the current bending status of the steerable distal section.

4. The medical system of claim 1, wherein the controller is further configured to assess a library of tools for passability through the tool channel and create a listing of the tools from the library of tools that are able to pass through the tool channel.

5. The medical system of claim 4, wherein the assessment of the listing of the tools is updated during navigation of the bendable body, wherein the controller is further configured to display the listing of the tools.

6. The medical system of claim 4, wherein assessing the library of tools is based on calculations from a pathway calculated by a planning software and a CT scan.

7. The medical system of claim 4, wherein the controller further assesses the library of tools for passability through the tool channel if the tool channel were switched to distributed bending mode.

8. The medical system of claim 4, wherein the library of tools comprises a plurality of tools select from biopsy needles, forceps, cryobiopsy probes, and/or cytology brushes.

9. The medical system of claim 1, wherein the distance D of a region of interest and/or the bend threshold Tis defined by the user.

10. The medical system of claim 1, wherein the bend threshold T is defined by the physical parameters of the steerable distal section.

11. The medical system of claim 1, wherein the bend status is defined by a shape sensor measurement, a wire force measurement, and/or a navigation plan.

12. The medical system of claim 1, wherein the first bending mode is a tip bending mode.

13. The robot system of claim 1, wherein the steerable distal section comprises at least a proximal and a distal steerable segment that are bendable by at least one proximal and at least one distal drive wire, and wherein, for distributed bending mode, the controller is configured to simultaneously move the at least one proximal drive wire and the at least one distal drive wire.

14. A method of assisting in the performance of a procedure using a bendable body comprising:
providing a bendable body for insertion into a patient;
in a first bending mode, autonomously navigating and/or receive instructions from a user for navigating towards a target while a camera is in the tool channel;
when the bendable body has reached within a distance D of the target and/or a bend status is above a bend threshold T, indicating the status to the user and autonomously changing to a distributed bending mode;
instructing the user to insert a tool and/or detecting that a tool has been inserted;
autonomously changing to the first bending mode.

15. The method of claim 14, further comprising:
verifying that the tip is pointing at the target after changing to distributed bending mode;
adjusting tip under image guidance after inserting tool; and
inserting camera after changing to first bending mode for navigation to a second site or removal of bendable body.

16. The method of claim 15, wherein the image guidance is CT image guidance, electromagnetic image guidance, or fluoroscopic image guidance.

17. The method of claim 14, further comprising the steps after performing the procedure of:
changing to distributed bending mode;
instructing the user to:
remove the tool,
detect that the tool has been removed,
insert the camera, and/or
detect that the camera has been inserted;
changing to a tip bending mode; and
autonomously navigating and/or receive instructions from a user for navigating towards a second target or removing the bendable body from the patient.

18. The method of claim 14, wherein the tool is a biopsy needle and the procedure is a biopsy procedure.

19. The method of claim 14, wherein the bend status is defined by a shape sensor measurement, a wire force measurement, and/or a navigation plan.

20. An information processing apparatus to control a bendable body;
at least one memory storing instructions; and
at least one processor that executes the instructions stored in the memory to act, based on user instruction, wherein the actions include:
navigating a bendable body that has been inserted into a patient towards a target in first bending mode with a camera in the tool channel;
changing to a distributed bending mode;
removing the camera;
inserting a tool;
changing to the first bending mode;
performing a procedure.
